SEQUENCE LISTING (245 sequences)

SEQ ID NO: 1 - protein, 15 aa, Artificial sequence
  D-amino acid peptide
  misc_feature (15)..(15): Xaa can be any naturally occurring amino acid
WWQSFSCHTVCQSFX

SEQ ID NO: 2 - protein, 14 aa, Artificial sequence
  D-amino acid peptide
WWQSFSCHTVCQSF

SEQ ID NO: 3 - protein, 13 aa, Artificial sequence
  D-amino acid peptide
WWQSFSCHTVCQS

SEQ ID NO: 4 - protein, 13 aa, Artificial sequence
  D-amino acid peptide
WQSFSCHTVCQSF

SEQ ID NO: 5 - protein, 11 aa, Artificial sequence
  D-amino acid peptide
QSFSCHTVCQS

SEQ ID NO: 6 - protein, 13 aa, Artificial sequence
  D-amino acid peptide
WWQSFSCHTVCQF

SEQ ID NO: 7 - protein, 12 aa, Artificial sequence
  D-amino acid peptide
WQSFSCHTVCQF

SEQ ID NO: 8 - protein, 11 aa, Artificial sequence
  D-amino acid peptide
QSFSCHTVCQF

SEQ ID NO: 9 - protein, 13 aa, Artificial sequence
  D-amino acid peptide
WWQSFSCHTVCSF

SEQ ID NO: 10 - protein, 12 aa, Artificial sequence
  D-amino acid peptide
WQSFSCHTVCSF

SEQ ID NO: 11 - protein, 11 aa, Artificial sequence
  D-amino acid peptide
QSFSCHTVCSF

SEQ ID NO: 12 - protein, 12 aa, Artificial sequence
  D-amino acid peptide
WWQSFSCHTVCQ

SEQ ID NO: 13 - protein, 11 aa, Artificial sequence
  D-amino acid peptide
WQSFSCHTVCQ

SEQ ID NO: 14 - protein, 10 aa, Artificial sequence
  D-amino acid peptide
QSFSCHTVCQ

SEQ ID NO: 15 - protein, 12 aa, Artificial sequence
  D-amino acid peptide
WWQSFSCHTVCS

SEQ ID NO: 16 - protein, 11 aa, Artificial sequence
  D-amino acid peptide
WQSFSCHTVCS

SEQ ID NO: 17 - protein, 10 aa, Artificial sequence
  D-amino acid peptide
QSFSCHTVCS

SEQ ID NO: 18 - protein, 12 aa, Artificial sequence
  D-amino acid peptide
WWQSFSCHTVCF

SEQ ID NO: 19 - protein, 11 aa, Artificial sequence
  D-amino acid peptide
WQSFSCHTVCF

SEQ ID NO: 20 - protein, 10 aa, Artificial sequence
  D-amino acid peptide
QSFSCHTVCF

SEQ ID NO: 21 - protein, 11 aa, Artificial sequence
  D-amino acid peptide
WWQSFSCHTVC

SEQ ID NO: 22 - protein, 10 aa, Artificial sequence
  D-amino acid peptide
WQSFSCHTVC

SEQ ID NO: 23 - protein, 9 aa, Artificial sequence
  D-amino acid peptide
QSFSCHTVC

SEQ ID NO: 24 - protein, 13 aa, Artificial sequence
  D-amino acid peptide
WQSFSCHTVCQSF

SEQ ID NO: 25 - protein, 12 aa, Artificial sequence
  D-amino acid peptide
QSFSCHTVCQSF

SEQ ID NO: 26 - protein, 21 aa, Artificial sequence
  D-amino acid peptide
SFSCHTVCQSFSFSCHTVCQS

SEQ ID NO: 27 - protein, 20 aa, Artificial sequence
  D-amino acid peptide
SFSCHTVCQFSFSCHTVCSF

SEQ ID NO: 28 - protein, 18 aa, Artificial sequence
  D-amino acid peptide
SFSCHTVCQSFSCHTVCS

SEQ ID NO: 29 - protein, 18 aa, Artificial sequence
  D-amino acid peptide
SFSCHTVCFSCHTVCQSF

SEQ ID NO: 30 - protein, 20 aa, Artificial sequence
  D-amino acid peptide
SCHTVCQSFWFSCHTVCQSF

SEQ ID NO: 31 - protein, 20 aa, Artificial sequence
  D-amino acid peptide
WSCHTVCQSFSSCHTVCQSF

SEQ ID NO: 32 - protein, 19 aa, Artificial sequence
  D-amino acid peptide
QSCHTVCQSFSCHTVCQSF

SEQ ID NO: 33 - protein, 24 aa, Artificial sequence
  D-amino acid peptide
QFSCHTVCQSFWWQSFSHTVCQSF

SEQ ID NO: 34 - protein, 25 aa, Artificial sequence
  D-amino acid peptide
WWQSFCHTVCQSFWWQSFHTVCQSF

SEQ ID NO: 35 - protein, 23 aa, Artificial sequence
  D-amino acid peptide
WQSFSHTVCQSFQSFSHTVCQSF

SEQ ID NO: 36 - protein, 23 aa, Artificial sequence
  D-amino acid peptide
WQSFCHTVCQSFQSFCHTVCQSF

SEQ ID NO: 37 - protein, 21 aa, Artificial sequence
  D-amino acid peptide
WQSFHTVCQSFQSFHTVCQSF

SEQ ID NO: 38 - protein, 24 aa, Artificial sequence
  D-amino acid peptide
WWQSFSTVCQSFWWQSFCTVCQSF

SEQ ID NO: 39 - protein, 12 aa, Artificial sequence
  D-amino acid peptide
WWQSFSHTVCQS

SEQ ID NO: 40 - protein, 12 aa, Artificial sequence
  D-amino acid peptide
WWQSFCHTVCQS

SEQ ID NO: 41 - protein, 11 aa, Artificial sequence
  D-amino acid peptide
WWQSFHTVCQS

SEQ ID NO: 42 - protein, 11 aa, Artificial sequence
  D-amino acid peptide
WWQSFSHTVCQ

SEQ ID NO: 43 - protein, 11 aa, Artificial sequence
  D-amino acid peptide
WWQSFCHTVCQ

SEQ ID NO: 44 - protein, 10 aa, Artificial sequence
  D-amino acid peptide
WWQSFHTVCQ

SEQ ID NO: 45 - protein, 10 aa, Artificial sequence
  D-amino acid peptide
WWQSFSHTVC

SEQ ID NO: 46 - protein, 10 aa, Artificial sequence
  D-amino acid peptide
WWQSFCHTVC

SEQ ID NO: 47 - protein, 9 aa, Artificial sequence
  D-amino acid peptide
WWQSFHTVC

SEQ ID NO: 48 - protein, 12 aa, Artificial sequence
  D-amino acid peptide
WWQSFSHTVCQF

SEQ ID NO: 49 - protein, 12 aa, Artificial sequence
  D-amino acid peptide
WWQSFCHTVCQF

SEQ ID NO: 50 - protein, 11 aa, Artificial sequence
  D-amino acid peptide
WWQSFHTVCQF

SEQ ID NO: 51 - protein, 12 aa, Artificial sequence
  D-amino acid peptide
WWQSFSHTVCSF

SEQ ID NO: 52 - protein, 12 aa, Artificial sequence
  D-amino acid peptide
WWQSFCHTVCSF

SEQ ID NO: 53 - protein, 11 aa, Artificial sequence
  D-amino acid peptide
WWQSFHTVCSF

SEQ ID NO: 54 - protein, 11 aa, Artificial sequence
  D-amino acid peptide
WWQSFSHTVCF

SEQ ID NO: 55 - protein, 11 aa, Artificial sequence
  D-amino acid peptide
WWQSFCHTVCF

SEQ ID NO: 56 - protein, 10 aa, Artificial sequence
  D-amino acid peptide
WWQSFHTVCF

SEQ ID NO: 57 - protein, 11 aa, Artificial sequence
  D-amino acid peptide
WWQSFSHTVCS

SEQ ID NO: 58 - protein, 11 aa, Artificial sequence
  D-amino acid peptide
WWQSFCHTVCS

SEQ ID NO: 59 - protein, 10 aa, Artificial sequence
  D-amino acid peptide
WWQSFHTVCS

SEQ ID NO: 60 - protein, 11 aa, Artificial sequence
  D-amino acid peptide
WQSFSHTVCQS

SEQ ID NO: 61 - protein, 11 aa, Artificial sequence
  D-amino acid peptide
WQSFCHTVCQS

SEQ ID NO: 62 - protein, 10 aa, Artificial sequence
  D-amino acid peptide
WQSFHTVCQS

SEQ ID NO: 63 - protein, 10 aa, Artificial sequence
  D-amino acid peptide
WQSFSHTVCQ

SEQ ID NO: 64 - protein, 10 aa, Artificial sequence
  D-amino acid peptide
WQSFCHTVCQ

SEQ ID NO: 65 - protein, 9 aa, Artificial sequence
  D-amino acid peptide
WQSFHTVCQ

SEQ ID NO: 66 - protein, 9 aa, Artificial sequence
  D-amino acid peptide
WQSFSHTVC

SEQ ID NO: 67 - protein, 9 aa, Artificial sequence
  D-amino acid peptide
WQSFCHTVC

SEQ ID NO: 68 - protein, 8 aa, Artificial sequence
  D-amino acid peptide
WQSFHTVC

SEQ ID NO: 69 - protein, 11 aa, Artificial sequence
  D-amino acid peptide
WQSFSHTVCQF

SEQ ID NO: 70 - protein, 11 aa, Artificial sequence
  D-amino acid peptide
WQSFCHTVCQF

SEQ ID NO: 71 - protein, 10 aa, Artificial sequence
  D-amino acid peptide
WQSFHTVCQF

SEQ ID NO: 72 - protein, 11 aa, Artificial sequence
  D-amino acid peptide
WQSFSHTVCSF

SEQ ID NO: 73 - protein, 11 aa, Artificial sequence
  D-amino acid peptide
WQSFCHTVCSF

SEQ ID NO: 74 - protein, 10 aa, Artificial sequence
  D-amino acid peptide
WQSFHTVCSF

SEQ ID NO: 75 - protein, 10 aa, Artificial sequence
  D-amino acid peptide
WQSFSHTVCF

SEQ ID NO: 76 - protein, 10 aa, Artificial sequence
  D-amino acid peptide
WQSFCHTVCF

SEQ ID NO: 77 - protein, 9 aa, Artificial sequence
  D-amino acid peptide
WQSFHTVCF

SEQ ID NO: 78 - protein, 10 aa, Artificial sequence
  D-amino acid peptide
WQSFSHTVCS

SEQ ID NO: 79 - protein, 10 aa, Artificial sequence
  D-amino acid peptide
WQSFCHTVCS

SEQ ID NO: 80 - protein, 9 aa, Artificial sequence
  D-amino acid peptide
WQSFHTVCS

SEQ ID NO: 81 - protein, 10 aa, Artificial sequence
  D-amino acid peptide
QSFSHTVCQS

SEQ ID NO: 82 - protein, 10 aa, Artificial sequence
  D-amino acid peptide
QSFCHTVCQS

SEQ ID NO: 83 - protein, 9 aa, Artificial sequence
  D-amino acid peptide
QSFHTVCQS

SEQ ID NO: 84 - protein, 9 aa, Artificial sequence
  D-amino acid peptide
QSFSHTVCQ

SEQ ID NO: 85 - protein, 10 aa, Artificial sequence
  D-amino acid peptide
WQSFCHTVCQ

SEQ ID NO: 86 - protein, 8 aa, Artificial sequence
  D-amino acid peptide
QSFHTVCQ

SEQ ID NO: 87 - protein, 8 aa, Artificial sequence
  D-amino acid peptide
QSFSHTVC

SEQ ID NO: 88 - protein, 8 aa, Artificial sequence
  D-amino acid peptide
QSFCHTVC

SEQ ID NO: 89 - protein, 7 aa, Artificial sequence
  D-amino acid peptide
QSFHTVC

SEQ ID NO: 90 - protein, 10 aa, Artificial sequence
  D-amino acid peptide
QSFSHTVCQF

SEQ ID NO: 91 - protein, 10 aa, Artificial sequence
  D-amino acid peptide
QSFCHTVCQF

SEQ ID NO: 92 - protein, 9 aa, Artificial sequence
  D-amino acid peptide
QSFHTVCQF

SEQ ID NO: 93 - protein, 10 aa, Artificial sequence
  D-amino acid peptide
QSFSHTVCSF

SEQ ID NO: 94 - protein, 10 aa, Artificial sequence
  D-amino acid peptide
QSFCHTVCSF

SEQ ID NO: 95 - protein, 9 aa, Artificial sequence
  D-amino acid peptide
QSFHTVCSF

SEQ ID NO: 96 - protein, 9 aa, Artificial sequence
  D-amino acid peptide
QSFSHTVCF

SEQ ID NO: 97 - protein, 9 aa, Artificial sequence
  D-amino acid peptide
QSFCHTVCF

SEQ ID NO: 98 - protein, 8 aa, Artificial sequence
  D-amino acid peptide
QSFHTVCF

SEQ ID NO: 99 - protein, 9 aa, Artificial sequence
  D-amino acid peptide
QSFSHTVCS

SEQ ID NO: 100 - protein, 9 aa, Artificial sequence
  D-amino acid peptide
QSFCHTVCS

SEQ ID NO: 101 - protein, 8 aa, Artificial sequence
  D-amino acid peptide
QSFHTVCS

SEQ ID NO: 102 - protein, 13 aa, Artificial sequence
  D-amino acid peptide
WWQSFSCHTVQSF

SEQ ID NO: 103 - protein, 13 aa, Artificial sequence
  D-amino acid peptide
WWQSFSCHTCQSF

SEQ ID NO: 104 - protein, 13 aa, Artificial sequence
  D-amino acid peptide
WWQSFSCHVCQSF

SEQ ID NO: 105 - protein, 12 aa, Artificial sequence
  D-amino acid peptide
WWQSFSCHTQSF

SEQ ID NO: 106 - protein, 12 aa, Artificial sequence
  D-amino acid peptide
WWQSFSCHVQSF

SEQ ID NO: 107 - protein, 12 aa, Artificial sequence
  D-amino acid peptide
WWQSFSCHCQSF

SEQ ID NO: 108 - protein, 11 aa, Artificial sequence
  D-amino acid peptide
WWQSFSCHQSF

SEQ ID NO: 109 - protein, 13 aa, Artificial sequence
  D-amino acid peptide
WWQSFSCTVCQSF

SEQ ID NO: 110 - protein, 12 aa, Artificial sequence
  D-amino acid peptide
WWQSFSCTVQSF

SEQ ID NO: 111 - protein, 12 aa, Artificial sequence
  D-amino acid peptide
WWQSFSCTCQSF

SEQ ID NO: 112 - protein, 12 aa, Artificial sequence
  D-amino acid peptide
WWQSFSCVCQSF

SEQ ID NO: 113 - protein, 11 aa, Artificial sequence
  D-amino acid peptide
WWQSFSCTQSF

SEQ ID NO: 114 - protein, 11 aa, Artificial sequence
  D-amino acid peptide
WWQSFSCVQSF

SEQ ID NO: 115 - protein, 11 aa, Artificial sequence
  D-amino acid peptide
WWQSFSCCQSF

SEQ ID NO: 116 - protein, 10 aa, Artificial sequence
  D-amino acid peptide
WWQSFSCQSF

SEQ ID NO: 117 - protein, 4 aa, Artificial sequence
  D-amino acid peptide
CQSF

SEQ ID NO: 118 - protein, 5 aa, Artificial sequence
  D-amino acid peptide
VCQSF

SEQ ID NO: 119 - protein, 6 aa, Artificial sequence
  D-amino acid peptide
TVCQSF

SEQ ID NO: 120 - protein, 7 aa, Artificial sequence
  D-amino acid peptide
HTVCQSF

SEQ ID NO: 121 - protein, 8 aa, Artificial sequence
  D-amino acid peptide
CHTVCQSF

SEQ ID NO: 122 - protein, 9 aa, Artificial sequence
  D-amino acid peptide
SCHTVCQSF

SEQ ID NO: 123 - protein, 10 aa, Artificial sequence
  D-amino acid peptide
FSCHTVCQSF

SEQ ID NO: 124 - protein, 11 aa, Artificial sequence
  D-amino acid peptide
SFSCHTVCQSF

SEQ ID NO: 125 - protein, 4 aa, Artificial sequence
  D-amino acid peptide
QSFC

SEQ ID NO: 126 - protein, 5 aa, Artificial sequence
  D-amino acid peptide
QSFSC

SEQ ID NO: 127 - protein, 4 aa, Artificial sequence
  D-amino acid peptide
QSFC

SEQ ID NO: 128 - protein, 8 aa, Artificial sequence
  D-amino acid peptide
WWQSFSCH

SEQ ID NO: 129 - protein, 7 aa, Artificial sequence
  D-amino acid peptide
WQSFSCH

SEQ ID NO: 130 - protein, 6 aa, Artificial sequence
  D-amino acid peptide
QSFSCH

SEQ ID NO: 131 - protein, 7 aa, Artificial sequence
  D-amino acid peptide
WWQSFCH

SEQ ID NO: 132 - protein, 6 aa, Artificial sequence
  D-amino acid peptide
WQSFCH

SEQ ID NO: 133 - protein, 5 aa, Artificial sequence
  D-amino acid peptide
QSFCH

SEQ ID NO: 134 - protein, 24 aa, Artificial sequence
  D-amino acid peptide
WWQSFSHTVQSFWWQSFCHTVQSF

SEQ ID NO: 135 - protein, 23 aa, Artificial sequence
  D-amino acid peptide
WWQSFHTVQSFWWQSFSHTCQSF

SEQ ID NO: 136 - protein, 23 aa, Artificial sequence
  D-amino acid peptide
WWQSFCHTCQSFWWQSFHTCQSF

SEQ ID NO: 137 - protein, 24 aa, Artificial sequence
  D-amino acid peptide
WWQSFSHVCQSFWWQSFCHVCQSF

SEQ ID NO: 138 - protein, 23 aa, Artificial sequence
  D-amino acid peptide
WWQSFHVCQSFWWQSFSTVCQSF

SEQ ID NO: 139 - protein, 23 aa, Artificial sequence
  D-amino acid peptide
WWQSFCTVCQSFWWQSFTVCQSF

SEQ ID NO: 140 - protein, 22 aa, Artificial sequence
  D-amino acid peptide
WWQSFSHTQSFWWQSFCHTQSF

SEQ ID NO: 141 - protein, 22 aa, Artificial sequence
  D-amino acid peptide
WWQSFHTQSFWWQSFSHVCQSF

SEQ ID NO: 142 - protein, 21 aa, Artificial sequence
  D-amino acid peptide
WWQSFCHVQSFWWQSFHVQSF

SEQ ID NO: 143 - protein, 22 aa, Artificial sequence
  D-amino acid peptide
WWQSFSHCQSFWWQSFCHCQSF

SEQ ID NO: 144 - protein, 20 aa, Artificial sequence
  D-amino acid peptide
WWQSFHCQSFWWQSFSHQSF

SEQ ID NO: 145 - protein, 19 aa, Artificial sequence
  D-amino acid peptide
WWQSFCHQSFWWQSFHQSF

SEQ ID NO: 146 - protein, 22 aa, Artificial sequence
  D-amino acid peptide
WWQSFSTVQSFWWQSFCTVQSF

SEQ ID NO: 147 - protein, 10 aa, Artificial sequence
  D-amino acid peptide
WWQSFTVQSF

SEQ ID NO: 148 - protein, 11 aa, Artificial sequence
  D-amino acid peptide
WWQSFSTCQSF

SEQ ID NO: 149 - protein, 11 aa, Artificial sequence
  D-amino acid peptide
WWQSFCTCQSF

SEQ ID NO: 150 - protein, 10 aa, Artificial sequence
  D-amino acid peptide
WWQSFTCQSF

SEQ ID NO: 151 - protein, 11 aa, Artificial sequence
  D-amino acid peptide
WWQSFSVCQSF

SEQ ID NO: 152 - protein, 11 aa, Artificial sequence
  D-amino acid peptide
WWQSFCVCQSF

SEQ ID NO: 153 - protein, 10 aa, Artificial sequence
  D-amino acid peptide
WWQSFVCQSF

SEQ ID NO: 154 - protein, 10 aa, Artificial sequence
  D-amino acid peptide
WWQSFSTQSF

SEQ ID NO: 155 - protein, 10 aa, Artificial sequence
  D-amino acid peptide
WWQSFCTQSF

SEQ ID NO: 156 - protein, 9 aa, Artificial sequence
  D-amino acid peptide
WWQSFTQSF

SEQ ID NO: 157 - protein, 10 aa, Artificial sequence
  D-amino acid peptide
WWQSFSVQSF

SEQ ID NO: 158 - protein, 10 aa, Artificial sequence
  D-amino acid peptide
WWQSFCVQSF

SEQ ID NO: 159 - protein, 9 aa, Artificial sequence
  D-amino acid peptide
WWQSFVQSF

SEQ ID NO: 160 - protein, 10 aa, Artificial sequence
  D-amino acid peptide
WWQSFSCQSF

SEQ ID NO: 161 - protein, 10 aa, Artificial sequence
  D-amino acid peptide
WWQSFCCQSF

SEQ ID NO: 162 - protein, 9 aa, Artificial sequence
  D-amino acid peptide
WWQSFCQSF

SEQ ID NO: 163 - protein, 9 aa, Artificial sequence
  D-amino acid peptide
WWQSFSQSF

SEQ ID NO: 164 - protein, 9 aa, Artificial sequence
  D-amino acid peptide
WWQSFCQSF

SEQ ID NO: 165 - protein, 8 aa, Artificial sequence
  D-amino acid peptide
WWQSFQSF

SEQ ID NO: 166 - protein, 11 aa, Artificial sequence
  D-amino acid peptide
WWQSFTVCQSF

SEQ ID NO: 167 - protein, 12 aa, Artificial sequence
  D-amino acid peptide
WWSFCHTVCQSF

SEQ ID NO: 168 - protein, 12 aa, Artificial sequence
  D-amino acid peptide
WWSFSHTVCQSF

SEQ ID NO: 169 - protein, 12 aa, Artificial sequence
  D-amino acid peptide
WWSFSCTVCQSF

SEQ ID NO: 170 - protein, 12 aa, Artificial sequence
  D-amino acid peptide
WWSFSCHVCQSF

SEQ ID NO: 171 - protein, 12 aa, Artificial sequence
  D-amino acid peptide
WWSFSCHTCQSF

SEQ ID NO: 172 - protein, 12 aa, Artificial sequence
  D-amino acid peptide
WWSFSCHTVQSF

SEQ ID NO: 173 - protein, 11 aa, Artificial sequence
  D-amino acid peptide
WWSFHTVCQSF

SEQ ID NO: 174 - protein, 11 aa, Artificial sequence
  D-amino acid peptide
WWSFCTVCQSF

SEQ ID NO: 175 - protein, 11 aa, Artificial sequence
  D-amino acid peptide
WWSFCHVCQSF

SEQ ID NO: 176 - protein, 11 aa, Artificial sequence
  D-amino acid peptide
WWSFCHTCQSF

SEQ ID NO: 177 - protein, 11 aa, Artificial sequence
  D-amino acid peptide
WWSFCHTVQSF

SEQ ID NO: 178 - protein, 11 aa, Artificial sequence
  D-amino acid peptide
WWSFSTVCQSF

SEQ ID NO: 179 - protein, 11 aa, Artificial sequence
  D-amino acid peptide
WWSFSHVCQSF

SEQ ID NO: 180 - protein, 11 aa, Artificial sequence
  D-amino acid peptide
WWSFSHTCQSF

SEQ ID NO: 181 - protein, 11 aa, Artificial sequence
  D-amino acid peptide
WWSFSHTVQSF

SEQ ID NO: 182 - protein, 11 aa, Artificial sequence
  D-amino acid peptide
WWSFSCVCQSF

SEQ ID NO: 183 - protein, 11 aa, Artificial sequence
  D-amino acid peptide
WWSFSCTCQSF

SEQ ID NO: 184 - protein, 11 aa, Artificial sequence
  D-amino acid peptide
WWSFSCTVQSF

SEQ ID NO: 185 - protein, 11 aa, Artificial sequence
  D-amino acid peptide
WWSFSCHCQSF

SEQ ID NO: 186 - protein, 11 aa, Artificial sequence
  D-amino acid peptide
WWSFSCHVQSF

SEQ ID NO: 187 - protein, 11 aa, Artificial sequence
  D-amino acid peptide
WWSFSCHTQSF

SEQ ID NO: 188 - protein, 12 aa, Artificial sequence
  D-amino acid peptide
WWQFCHTVCQSF

SEQ ID NO: 189 - protein, 12 aa, Artificial sequence
  D-amino acid peptide
WWQFSHTVCQSF

SEQ ID NO: 190 - protein, 12 aa, Artificial sequence
  D-amino acid peptide
WWQFSCTVCQSF

SEQ ID NO: 191 - protein, 12 aa, Artificial sequence
  D-amino acid peptide
WWQFSCHVCQSF

SEQ ID NO: 192 - protein, 12 aa, Artificial sequence
  D-amino acid peptide
WWQFSCHTCQSF

SEQ ID NO: 193 - protein, 12 aa, Artificial sequence
  D-amino acid peptide
WWQFSCHTVQSF

SEQ ID NO: 194 - protein, 11 aa, Artificial sequence
  D-amino acid peptide
WWQFHTVCQSF

SEQ ID NO: 195 - protein, 11 aa, Artificial sequence
  D-amino acid peptide
WWQFCTVCQSF

SEQ ID NO: 196 - protein, 11 aa, Artificial sequence
  D-amino acid peptide
WWQFCHVCQSF

SEQ ID NO: 197 - protein, 11 aa, Artificial sequence
  D-amino acid peptide
WWQFCHTCQSF

SEQ ID NO: 198 - protein, 11 aa, Artificial sequence
  D-amino acid peptide
WWQFCHTVQSF

SEQ ID NO: 199 - protein, 11 aa, Artificial sequence
  D-amino acid peptide
WWQFSTVCQSF

SEQ ID NO: 200 - protein, 11 aa, Artificial sequence
  D-amino acid peptide
WWQFSHVCQSF

SEQ ID NO: 201 - protein, 11 aa, Artificial sequence
  D-amino acid peptide
WWQFSHTCQSF

SEQ ID NO: 202 - protein, 11 aa, Artificial sequence
  D-amino acid peptide
WWQFSHTVQSF

SEQ ID NO: 203 - protein, 11 aa, Artificial sequence
  D-amino acid peptide
WWQFSCVCQSF

SEQ ID NO: 204 - protein, 11 aa, Artificial sequence
  D-amino acid peptide
WWQFSCTCQSF

SEQ ID NO: 205 - protein, 11 aa, Artificial sequence
  D-amino acid peptide
WWQFSCTVQSF

SEQ ID NO: 206 - protein, 11 aa, Artificial sequence
  D-amino acid peptide
WWQFSCHCQSF

SEQ ID NO: 207 - protein, 11 aa, Artificial sequence
  D-amino acid peptide
WWQFSCHVQSF

SEQ ID NO: 208 - protein, 11 aa, Artificial sequence
  D-amino acid peptide
WWQFSCHTQSF

SEQ ID NO: 209 - protein, 12 aa, Artificial sequence
  D-amino acid peptide
WWQSCHTVCQSF

SEQ ID NO: 210 - protein, 12 aa, Artificial sequence
  D-amino acid peptide
WWQSSHTVCQSF

SEQ ID NO: 211 - protein, 12 aa, Artificial sequence
  D-amino acid peptide
WWQSSCTVCQSF

SEQ ID NO: 212 - protein, 12 aa, Artificial sequence
  D-amino acid peptide
WWQSSCHVCQSF

SEQ ID NO: 213 - protein, 12 aa, Artificial sequence
  D-amino acid peptide
WWQSSCHTCQSF

SEQ ID NO: 214 - protein, 12 aa, Artificial sequence
  D-amino acid peptide
WWQSSCHTVQSF

SEQ ID NO: 215 - protein, 11 aa, Artificial sequence
  D-amino acid peptide
WWQSHTVCQSF

SEQ ID NO: 216 - protein, 11 aa, Artificial sequence
  D-amino acid peptide
WWQSCTVCQSF

SEQ ID NO: 217 - protein, 11 aa, Artificial sequence
  D-amino acid peptide
WWQSCHVCQSF

SEQ ID NO: 218 - protein, 11 aa, Artificial sequence
  D-amino acid peptide
WWQSCHTCQSF

SEQ ID NO: 219 - protein, 11 aa, Artificial sequence
  D-amino acid peptide
WWQSCHTVQSF

SEQ ID NO: 220 - protein, 11 aa, Artificial sequence
  D-amino acid peptide
WWQSSTVCQSF

SEQ ID NO: 221 - protein, 11 aa, Artificial sequence
  D-amino acid peptide
WWQSSHVCQSF

SEQ ID NO: 222 - protein, 11 aa, Artificial sequence
  D-amino acid peptide
WWQSSHTCQSF

SEQ ID NO: 223 - protein, 11 aa, Artificial sequence
  D-amino acid peptide
WWQSSHTVQSF

SEQ ID NO: 224 - protein, 11 aa, Artificial sequence
  D-amino acid peptide
WWQSSCVCQSF

SEQ ID NO: 225 - protein, 11 aa, Artificial sequence
  D-amino acid peptide
WWQSSCTCQSF

SEQ ID NO: 226 - protein, 11 aa, Artificial sequence
  D-amino acid peptide
WWQSSCTVQSF

SEQ ID NO: 227 - protein, 11 aa, Artificial sequence
  D-amino acid peptide
WWQSSCHCQSF

SEQ ID NO: 228 - protein, 11 aa, Artificial sequence
  D-amino acid peptide
WWQSSCHVQSF

SEQ ID NO: 229 - protein, 11 aa, Artificial sequence
  D-amino acid peptide
WWQSSCHTQSF

SEQ ID NO: 230 - protein, 20 aa, Artificial sequence
  D-amino acid peptide
WWCHTVCQSFWWSHTVCQSF

SEQ ID NO: 231 - protein, 20 aa, Artificial sequence
  D-amino acid peptide
WWSCTVCQSFWWSCHVCQSF

SEQ ID NO: 232 - protein, 20 aa, Artificial sequence
  D-amino acid peptide
WWSCHTCQSFWWSCHTVQSF

SEQ ID NO: 233 - protein, 18 aa, Artificial sequence
  D-amino acid peptide
WWHTVCQSFWWCTVCQSF

SEQ ID NO: 234 - protein, 18 aa, Artificial sequence
  D-amino acid peptide
WWCHVCQSFWWCHTCQSF

SEQ ID NO: 235 - protein, 18 aa, Artificial sequence
  D-amino acid peptide
WWCHTVQSFWWSTVCQSF

SEQ ID NO: 236 - protein, 18 aa, Artificial sequence
  D-amino acid peptide
WWSHVCQSFWWSHTCQSF

SEQ ID NO: 237 - protein, 18 aa, Artificial sequence
  D-amino acid peptide
WWSHTVQSFWWSCVCQSF

SEQ ID NO: 238 - protein, 18 aa, Artificial sequence
  D-amino acid peptide
WWSCTCQSFWWSCTVQSF

SEQ ID NO: 239 - protein, 18 aa, Artificial sequence
  D-amino acid peptide
WWSCHCQSFWWSCHVQSF

SEQ ID NO: 240 - protein, 18 aa, Artificial sequence
  D-amino acid peptide
WWSCHTQSFWWSHTCQSF

SEQ ID NO: 241 - protein, 17 aa, Artificial sequence
  D-amino acid peptide
WWCHTCQSFWWHTCQSF

SEQ ID NO: 242 - protein, 18 aa, Artificial sequence
  D-amino acid peptide
WWCHTVQSFWWSHTVQSF

SEQ ID NO: 243 - protein, 16 aa, Artificial sequence
  D-amino acid peptide
WWHTVQSFWWSHTQSF

SEQ ID NO: 244 - protein, 15 aa, Artificial sequence
  D-amino acid peptide
WWCHTQSFWWHTQSF

SEQ ID NO: 245 - protein, 28 aa, Artificial sequence
  D-amino acid peptide
WWQSFSCHTVCQSFWWQSFSCHTVCQSF

(19) United States
(12) Patent Application Publication
(10) Pub. No.: US 2021/0236597 A1
(43) Pub. Date: Aug. 5, 2021

(54) METHODS AND COMPOSITIONS FOR TREATING AND DIAGNOSING POLYCYSTIC OVARY SYNDROME

(71) Applicant: The Board of Regents of the University of Oklahoma, Norman, OK (US)

(72) Inventor: David C. KEM, Edmond, OK (US)

(21) Appl. No.: 17/165,070

(22) Filed: Feb. 2, 2021

Related U.S. Application Data
(60) Provisional application No. 62/969,880, filed on Feb. 4, 2020.

(51) Int. Cl. A61K 38/17; C07K 14/72; G01N 33/566

(52) U.S. Cl. CPC A61K 38/1796; G01N 2800/36; G01N 33/566; C07K 14/723

(57) ABSTRACT

Compounds, compositions, and delivery devices, for treating and diagnosing conditions and diseases associated with activation of the gonadotropin releasing hormone receptor (GnRHR), particularly those involving GnRHR activating autoantibodies (GnRHR AAbs). In one non-limiting embodiment, the disease is Polycystic Ovary Syndrome (PCOS). The therapeutic compounds in at least certain non-limiting embodiments include peptides which comprise D-amino acids (D-amino acid peptides) which are able to bind with high affinity to GnRHR AAbs.

### CROSS-REFERENCE TO RELATED APPLICATIONS

This application claims benefit under 35 U.S.C. § 119(e) of U.S. Provisional Application Ser. No. 62/969,880, filed Feb. 4, 2020. The entire contents of the above-referenced patent application(s) are hereby expressly incorporated herein by reference.

### BACKGROUND

Polycystic Ovary Syndrome (PCOS) is a systemic disease characterized by multiple ovarian cysts, infertility, pain, discomfort, and associated metabolic abnormalities including insulin resistant diabetes mellitus, abnormal sex hormone function, hirsutism, and acne. It is considered of unknown etiology and frequently occurs in adolescence. PCOS involves the pituitary-ovarian reproductive organs, resulting in anovulatory infertility in 8-10% of women of reproductive age. Its causation is currently unknown but is associated with abnormal pituitary control and/or release of both luteinizing hormone (LH) and follicle stimulating hormone (FSH), both of which are intimately related to development and release of ova from the ovary in timely fashion for possible fertilization when a viable sperm is in proximity. In P C O S, the release of the developing ova is severely impaired, and ova are generally internally reabsorbed without release. The developing follicle continues to be stimulated by this abnormal absence of rupture and release and becomes a persistent and enlarged cyst, leading to distortion of the ovarian architecture and further disturbing normal development of subsequent follicles.

This infertility is profound in many conditions, leading to heartbreak, frustration, self-recrimination, and marital discord. Current infertility therapies have a poor success rate, and the current state of the art resides in in vitro fertilization, often requiring a separate donor and emplacement of the embryo. The costs are very high (e.g., $75K for three attempts) and are without guarantee of issue. Moreover, PCOS is not just an issue of infertility. A significant majority of women with PCOS develop a complex syndrome over several years that includes obesity, significant insulin resistance manifested by Type 2 diabetes mellitus, hypertension, and signs of hyper-androgenism (elevation of testosterone and weak androgens) including acne and hirsutism. A smaller subgroup includes women of thin nature who, despite eating freely, do not develop the above-mentioned metabolic syndrome. These women nevertheless share in the infertility and polycystic ovary presentation. This metabolic syndrome that is so prevalent must be treated using multiple dietary and pharmacological approaches. Women with PCOS who have diabetes have most commonly been treated with metformin with modest improvement, despite the frequent side effects of the medication and its long term inability to cure the disease. Interestingly, in some subjects there is minimal or very modest improvement in the infertility, but this is not common. Newer diabetes medications are being tried but have significant costs and are not recognized as more effective than metformin on the infertility. The hypertension is treated with usually more than one antihypertensive agent with their concomitant cost and side effects. The hyper-androgen state is of particular concern for women of all ages but is especially devastating for young women in society. The therapy usually is started too late and/or is inadequate and consists of using birth control pill (BCP) therapy to suppress LH and the use of androgen receptor antagonists with their concurrent side effects and expense. Improved methods and compositions for diagnosis and treatment of PCOS are highly desired. It is to meeting these needs that the present disclosure is directed.

### DETAILED DESCRIPTION

The present disclosure is directed, in at least certain non-limiting embodiments, to compositions and methods of treating and diagnosing conditions and diseases associated with activation of the gonadotropin releasing hormone receptor (GnRHR), particularly (but not limited to) those involving GnRHR activating autoantibodies (GnRHR AAbs). A particular (but non-limiting) feature of the present disclosure is the discovery of the relationship of GnRHR AAbs and Polycystic Ovary Syndrome (PCOS). Therefore, in at least one embodiment, the present disclosure is directed to treatment of PCOS by the inactivation of GnRHR AAbs. The therapeutic compounds in at least certain non-limiting embodiments include peptides that completely or partially comprise D-amino acids, and in further embodiments include retro-inverso D-amino acid (RID) peptides, which are able to bind with high affinity to GnRHR AAbs, thereby inhibiting the binding of the GnRHR AAbs to GnRHR by occupying the GnRHR-binding site of the GnRHR AAbs.

Without wishing to be bound by theory, it is believed that activating autoantibodies (GnRHR AAbs) which bind to the extracellular loop 2 (ECL2) of GnRHR are produced from an extraneous stimulus (e.g., an infection, stress, etc.) and interact with hypothalamic/pituitary GnRH receptors to inappropriately alter the synthesis and periodic release of LH, leading to hyperandrogenemia, induction of insulin resistance (IR), and metabolic complications.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. Thus, while the compounds, compositions, and methods of the present disclosure have been described in terms of particular (but non-limiting) embodiments, it will be apparent to those of skill in the art that variations may be applied to the compounds, compositions, and methods, and in the steps or in the sequence of steps of the methods described herein without departing from the concept, spirit, and scope of the inventive concepts.

All patents, patent applications, and non-patent publications including published articles mentioned in the specification or referenced in any portion of this application including (but not limited to) U.S. Provisional Application Ser. No. 62/634,324, filed Feb. 23, 2018; U.S. patent application Ser. No. 16/274,944, filed on Feb. 13, 2019, and published as U.S. Pat. App. Pub. 2019/0263862; and U.S. Provisional Application Ser. No. 62/969,880, filed Feb. 4, 2020, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities, and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are for purposes of illustration only and are to be construed as non-limiting of the claims.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

By “biologically active” is meant the ability to modify the physiological system of an organism without reference to how the active agent has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals or subjects within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic or treatment effect in a subject without excessive adverse side effects (such as (but not limited to) substantial toxicity, irritation, and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

The following abbreviations may be used herein for amino acids: alanine:ala:A; arginine:arg:R; asparagine:asn:N; aspartic acid:asp:D; cysteine:cys:C; glutamic acid:glu:E; glutamine:gln:Q; glycine:gly:G; histidine:his:H; isoleucine:ile:I; leucine:leu:L; lysine:lys:K; methionine:met:M; phenylalanine:phe:F; proline:pro:P; serine:ser:S; threonine:thr:T; tryptophan:trp:W; tyrosine:tyr:Y; and valine:val:V.

The term “peptide” is used herein to designate a series of amino acid residues, connected one to the other typically by peptide bonds between the alpha-amino and carbonyl groups of the adjacent amino acids to form an amino acid sequence. In certain non-limiting embodiments, the peptides can range in length from 4 to 10 to 15 to 25 to 40 to 60 to 75 to 100 amino acids, for example, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids.

The term “polypeptide” or “protein” is used herein to designate a series of amino acid residues, connected one to the other typically by peptide bonds between the alpha-amino and carbonyl groups of the adjacent amino acids, wherein the length is longer than a single peptide. A peptide conjugate refers, in a non-limiting embodiment, to a compound comprising a peptide of the present disclosure which is conjugated (e.g., covalently linked, directly or indirectly via a linker sequence) to another molecule, such as (but not limited to) a carrier molecule such as (but not limited to) a protein or other polymeric molecule, e.g., a serum albumin molecule or antibody, or other therapeutic compound such as (but not limited to) a drug, or an imaging or diagnostic moiety, and wherein the peptide retains its activity (e.g., binding, targeting, imaging, or inhibitory) even when conjugated to the molecule. The peptides of the present disclosure may be produced using any nucleotide sequence which encodes the desired amino acid sequence. Any of the peptides described herein or active variants thereof may be used to make the peptide conjugates of the present disclosure.

Peptides of the present disclosure and the nucleic acids which encode them include peptide and nucleic acid variants which comprise substitutions (conservative or non-conservative) of the native amino acids or bases. For example, the peptide variants include, but are not limited to, variants that are not exactly the same as the sequences disclosed herein, but which have, in addition to the substitutions explicitly described for various sequences listed herein, additional substitutions of amino acid residues (conservative or non-conservative) which substantially do not impair the activity or properties of the variants described herein. Examples of such conservative amino acid substitutions may include, but are not limited to, ala to gly, ser, or thr; arg to gln, his, or lys; asn to asp, gln, his, lys, ser, or thr; asp to asn or glu; cys to ser; gln to arg, asn, glu, his, lys, or met; glu to asp, gln, or lys; gly to pro or ala; his to arg, asn, gln, or tyr; ile to leu, met, or val; leu to ile, met, phe, or val; lys to arg, asn, gln, or glu; met to gln, ile, leu, or val; phe to leu, met, trp, or tyr; ser to ala, asn, met, or thr; thr to ala, asn, ser, or met; trp to phe or tyr; tyr to his, phe or trp; and val to ile, leu, or met.

One of ordinary skill in the art would readily know how to make, identify, select, or test such variants for receptor targeting activity against the same receptors targeted by the native peptides. Particular examples of conservative amino acid substitutions include, but are not limited to, gly:ala substitutions; val:ile:leu substitutions; asn:glu:his substitutions; asp:glu substitutions; ser:thr:met substitutions; lys:arg:his substitutions; and phe:tyr:trp substitutions. Other types of substitutions, variations, additions, deletions, and derivatives that result in functional variant peptides are also encompassed by the present disclosure, and one of skill in the art would readily know how to make, identify, or select such variants or derivatives, and how to test for receptor binding activity of those variants.

The term “homologous” or “% identity” as used herein means a nucleic acid (or fragment thereof) or a peptide having a degree of homology to the corresponding natural reference nucleic acid or peptide that may be in excess of 60%, or in excess of 65%, or in excess of 70%,or in excess of 75%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%, or other specific percentages described herein. For example, in regard to peptides, the percentage of homology or identity as described herein is typically calculated as the percentage of amino acid residues found in the smaller of the two sequences which align with identical amino acid residues in the sequence being compared, when four gaps per 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross-reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

In at least one embodiment, “% identity” represents the number of amino acids or nucleotides which are identical at corresponding positions in two sequences of a peptide or nucleic acids encoding similar peptides. For example, two amino acid sequences each having 15 residues will have at least 60% identity when at least 9 of the amino acids at corresponding positions are the same, at least 66% identity when at least 10 of the amino acids at corresponding positions are the same, at least 73% identity when at least 11 of the amino acids at corresponding positions are the same, at least 80% identity when at least 12 of the amino acids at corresponding positions are the same, at least 86% identity when at least 13 of the amino acids at corresponding positions are the same, and at least 93% identity when at least 14 of the amino acids at corresponding positions are the same. In another example, two amino acid sequences each having 19 residues will have at least 73% identity when at least 14 of the amino acids at corresponding positions are the same, at least 78% identity when at least 15 of the amino acids at corresponding positions are the same, at least 84% identity when at least 16 of the amino acids at corresponding positions are the same, at least 89% identity when at least 17 of the amino acids at corresponding positions are the same, and at least 94% identity when at least 18 of the amino acids at corresponding positions are the same.

Similarly, two amino acid sequences each having 20 residues will have at least 95% identity when 19 of the amino acids at corresponding positions are the same, or at least 90% identity when at least 18 of the amino acids at corresponding positions are the same, or at least 85% identity when at least 17 of the amino acids at corresponding positions are the same, or at least 80% identity when at least 16 of the amino acids at corresponding positions are the same. In other non-limiting examples, two amino acid sequences each having 100 residues will have 95% identity when 95 of the amino acids at corresponding positions are the same. Two amino acid sequences each having 100 residues will have at least 90% identity when at least 90 of the amino acids at corresponding positions are the same. Further, where a sequence is described herein as having “at least X % identity to” a reference sequence, this is intended to include, unless indicated otherwise, all percentages greater than X %, such as for example, (X+1)%, (X+2)%, (X+3)%, (X+4)%, and so on, up to 100%.

In at least certain non-limiting embodiments, the present disclosure is directed to a peptide comprising 4 to 45 amino acids, wherein the 4 to 45 amino acids comprise a retro-inverso amino acid sequence comprising D-amino acids. The peptide may be disposed in a pharmaceutically-acceptable carrier, vehicle, and/or diluent.

In at least certain non-limiting embodiments, the present disclosure is directed to peptides which comprise D-amino acids and may be referred to herein as D-peptides. The D-peptides may be from 4 to 45 amino acids long, and may comprise the amino acid sequence W₁W₂Q₃S₄F₅S₆C₇H₈T₉V₁₀C₁₁Q₁₂S₁₃F₁₄X₁₅ (SEQ ID NO:1), wherein X₁₅ is either C or is absent. When X₁₅ is absent, 1, 2, 3, 4, 5, 6, 7, 8, 9, or 10 of the D-amino acids W₁, W₂, Q₃, S₄, F₅, S₆, C₇, H₈, T₉, V₁₀, C₁₁, Q₁₂, S₁₃, and F₁₄ may be deleted, either contiguously or non-contiguously (for non-limiting examples of such deletions refer to SEQ ID NOS:2-245 in Tables 1-13). When X₁₅ is C, zero, 1, 2, 3, 4, 5, 6, 7, 8, 9, 10, or 11 of the D-amino acids W₁, W₂, Q₃, S₄, F₅, S₆, C₇, H₈, T₉, V₁₀, C₁₁, Q₁₂, S₁₃, and F₁₄ may be deleted, either contiguously or non-contiguously. Example of deletions when X₁₅ is either C or is absent include, but are not limited to, Q₁₂, S₁₃, and F₁₄; W₁ and W₂; W₁, W₂, Q₃, S₄, and F₅; C₇; C₁₁; V₁₀ and C₁₁; H₈, T₉, V₁₀, and C₁₁; S₆ and C₇; S₆, C₇, H₈, and T₉; S₆, C₇, H₈, T₉, V₁₀, and C₁₁; W₁, W₂, Q₃, S₄, F₅, S₆, C₇, H₈, T₉, and V₁₀; and W₁, W₂, Q₃, S₄, F₅, S₆, C₇, H₈, T₉, V₁₀, and C₁₁ when X₁₅ is C.

In certain particular (but non-limiting) embodiments, the peptide is selected from the group consisting of SEQ ID NOS:1-245.

In certain particular (but non-limiting) embodiments, the peptide binds to a gonadotropin releasing hormone receptor activating autoantibody (GnRHR-AAb) at an epitope which reduces binding of the GnRHR-AAb to the extracellular loop 2 (ECL2) of GnRHR.

The D-amino acid peptides can be synthesized using the technology disclosed in Patent Application Publication No. US 2016/0222084, published Aug. 4, 2016 (U.S. Ser. No. 14/776,855), which is hereby expressly incorporated herein by reference in its entirety.

In at least certain non-limiting embodiments, the present disclosure is directed to a drug delivery device or composition that comprises one or more of any of the peptides described or otherwise contemplated herein. In certain particular (but non-limiting) embodiments, the drug delivery device or composition substantially releases the one or more peptides over an extended period in a range of from about 6 hours to about 120 days.

In at least certain non-limiting embodiments, the present disclosure is directed to a method of treating a disease or condition associated with the gonadotropin releasing hormone receptor (GnRHR), comprising the step of: administering to a subject in need of such therapy at least one of any of the peptides described herein or otherwise contemplated herein. The peptide may be administered in a composition comprising a pharmaceutically-acceptable carrier, vehicle, and/or diluent. In at least one embodiment of the method, the disease or condition is Polycystic Ovary Syndrome (PCOS). In at least one embodiment of the method, the disease is a condition directly related to (consequent to) Polycystic Ovary Syndrome. Non-limiting examples of conditions in male and female subjects which can be treated by the presently disclosed peptides and peptide compositions include (but are not limited to) GnRHR-AAb-positive Metabolic Syndrome with abnormal LH and/or testosterone levels, insulin resistance, diabetes mellitus, elevated lipids, and elevated cardiovascular risk. In at least one embodiment of the method, the disease or condition is a refractory cancer selected from the group consisting of ovarian, breast, endometrial, uterine, prostate, and testicular cancer.

In at least certain non-limiting embodiments, the present disclosure is directed to an assay for detecting a gonadotropin releasing hormone receptor activating autoantibody (GnRHR AAb), comprising: a test surface having any of the peptides disclosed or otherwise contemplated herein bound thereto (such as, but not limited to a peptide comprising an amino acid sequence of any of SEQ ID NOS:1-245), or a portion thereof able to bind with high affinity and specificity to the GnRHR AAb, or a reverse amino acid sequence of the peptides listed in SEQ ID NOS:1-245 and/or described in any one of Tables 1-13, wherein the reverse amino acid sequences comprise L-amino acids.

In certain non-limiting embodiments, D-amino acid peptides (D-peptides) of the present disclosure, such as shown in Tables 1-13 can be used to preferentially bind to the GnRHR-AAbs forming a GnRHR-AAb-D-peptide complex, causing the AAb-D-peptide complex to be cleared by the R-E cells lining the liver and the spleen to engulf the complex and eliminate it from the circulation. The D-peptide thus is a decoy peptide that draws down the GnRHR-AAb levels and reduces or prevents the GnRHR-AAb from reaching and binding to the GnRHR receptor.

The D-amino acid-containing peptides of the present disclosure possess many advantages over the prior art, including but not limited to: (1) the ability to withstand all natural peptidases which can act on peptides comprising L-amino acids; (2) the capability for withstanding gastro-enteric exposure to natural peptidases and to limited acid hydrolysis; and (3) the potential for oral administration.

The compositions of the present disclosure may further contain a conjugate of any of the D-amino acid-containing peptides disclosed, described, or otherwise contemplated herein associated with a labeling agent. Various methods of labeling peptides are known in the art and may be used in accordance with the present disclosure. Examples of labels for polypeptides include, but are not limited to, the following: radioisotopes or radionuclides, fluorescent labels, chemiluminescent labels, and the like. In some embodiments, the labeling agent may be attached to the peptide by a spacer arm of various lengths to reduce potential steric hindrance. In addition, the terms “label,” “labeling agent,” “detectable marker,” “detection moiety,” and “reporter molecule” are used interchangeably herein. These conjugates are useful in various diagnostic methods, as discussed in more detail elsewhere herein.

Therapeutic Uses

D-peptides of the present disclosure, such as but not limited to those having sequences shown in Tables 1-13 and listed in SEQ ID NOS:1-245, may be administered in therapeutically effective amounts to treat the various conditions identified herein which are associated with GnRHR, such as (but not limited to) PCOS. An effective amount is a dosage of the composition sufficient to provide a therapeutically or medically desirable result or effect in the subject to which the composition is administered. The effective amount will vary with the particular condition being treated, the age and physical condition of the subject being treated, the severity of the condition, the duration of the treatment, the nature of the concurrent or combination therapy (if any), the specific route of administration, and like factors within the knowledge and expertise of the health practitioner. For example, in connection with methods directed towards treating subjects having a condition characterized by PCOS, an effective amount would be an amount sufficient to mitigate, reduce, modulate, inhibit, or otherwise effectively treat one or more of the conditions in the subject which are associated with PCOS, including (but not limited to) obesity, insulin resistance as manifested by Type 2 diabetes mellitus, hypertension, and hyper-androgenism (elevation of testosterone and weak androgens) including acne and hirsutism.

GnRHRs are known to be present in peripheral tissues of the female reproductive tract, such as (but not limited to) the ovaries and endometrium. Over 50% of resistant tumors of the breast and prostate are also reported to harbor GnRHR. Therefore, in at least certain non-limiting embodiments, the present disclosure is directed to the treatment of cancers, particularly (but not limited to) refractory cancers, associated with the presence of GnRHR-activating autoantibodies, including (but not limited to) breast, ovarian, endometrial, uterine, testicular, and prostate cancers. This technology permits development of patient selective (precise) therapeutic removal of the deleterious autoantibodies. The AAbs can be cleared from the systemic circulation by the GnRHR AAb-binding peptides described elsewhere herein.

Generally, a therapeutically effective amount of a peptide will vary with the subject's age, condition, and sex, as well as the nature and extent of the disease in the subject, all of which can be determined by one of ordinary skill in the art. The dosage may be adjusted by the individual physician or veterinarian, particularly in the event of any complication. A therapeutically effective amount is typically, but not limited to, an amount in a range from about 0.1 μg/kg to about 2000 mg/kg, or from about 1.0 μg/kg to about 1000 mg/kg, or from about 0.1 mg/kg to about 500 mg/kg, or from about 1.0 mg/kg to about 100 mg/kg, in one or more dose administrations daily, for one or more days. If desired, the effective daily dose of the active compound may be administered as two, three, four, five, six, or more sub-doses, for example, administered separately at appropriate intervals throughout the day, optionally, in unit dosage forms. In some embodiments, the compositions are administered for more than 7 days, more than 10 days, more than 14 days, or more than 20 days. In still other embodiments, the peptide is administered over a period of weeks or months. In still other embodiments, the peptide is delivered on alternate days, for example, the agent may be delivered every two days, or every three days, or every four days, or every five days, or every six days, or every week, or every month.

The peptide compositions may be administered alone or in combination with the above-described drug therapies and may be administered by a variety of administration routes. The particular mode selected will depend upon the compound selected, the condition being treated, the severity of the condition, whether the treatment is therapeutic or prophylactic, and the dosage required for efficacy. The methods of the present disclosure, generally speaking, may be practiced using any mode of administration that is medically acceptable, meaning any mode that produces effective levels of the active compounds without causing clinically unacceptable adverse effects. The administration may, for example, be oral, intraperitoneal, intra-cavity such as (but not limited to) rectal or vaginal, transdermal, topical, nasal, inhalation, mucosal, interdermal, or parenteral routes. The term “parenteral” includes subcutaneous, intravenous, intramuscular, or infusion. Intravenous or intramuscular routes may not be particularly suitable for long term therapy and prophylaxis. In certain non-limiting embodiments, however, it may be appropriate to administer the compound in a continuous infusion every several days, or once a week, or every several weeks, or once a month. Intravenous or intramuscular routes may be particularly used in emergency situations. Oral administration may be used for prophylactic treatment because of the convenience to the patient as well as the dosing schedule. Likewise, sustained release devices may be useful in certain non-limiting embodiments for prophylactic or post-surgery treatment, for example.

Particular pharmaceutical formulations of the presently disclosed peptide compositions include, but are not limited to, sterile aqueous or non-aqueous solutions, suspensions, and emulsions. Non-limiting examples of non-aqueous solvents include propylene glycol, polyethylene glycol, vegetable oils such as (but not limited to) olive oil, and injectable organic esters such as (but not limited to) ethyl oleate. Aqueous carriers include water, alcoholic/aqueous solutions, emulsions, or suspensions, including saline and buffered media. Parenteral vehicles include, for example, sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example (but not by way of limitation), antimicrobials, anti-oxidants, chelating compounds, inert gases, and the like. The pharmaceutical compositions may conveniently be presented in unit dosage form and may be prepared by any of the methods well known in the art of pharmacy.

Compositions suitable for oral administration may comprise discrete units, such as (but not limited to) capsules, tablets, and lozenges, each containing a predetermined amount of the peptide composition. Other compositions include suspensions in aqueous liquids or non-aqueous liquids such as (but not limited to) syrup, an elixir, or an emulsion. In yet other embodiments, the particular vehicle is a biocompatible microparticle or implant that is suitable for implantation into the mammalian recipient.

Other embodiments of the peptide compositions include pharmaceutically acceptable compositions which comprise a therapeutically-effective amount of one or more of the compounds described herein, formulated together with one or more pharmaceutically acceptable carriers (additives) and/or diluents. As described in detail herein, the pharmaceutically acceptable compositions may be specially formulated for administration in solid or liquid form, including, but not limited to, those adapted for the following: oral administration, for example, aqueous or non-aqueous solutions or suspensions, tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin; intravaginal or intrarectal administration, for example, as a cream or foam; sublingual administration; ocular administration; transdermal administration; or nasal administration.

Some examples of materials which can serve as pharmaceutically-acceptable carriers include, but are not limited to: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

In solid dosage forms of the peptide compositions for oral administration (capsules, tablets, pills, powders, granules, and the like), the compound or compounds may be mixed with one or more pharmaceutically-acceptable carriers, including, but not limited to, sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as cetyl alcohol, glycerol monostearate, and non-ionic surfactants; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets, and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Liquid dosage forms for oral administration of the peptides include, but are not limited to, pharmaceutically acceptable emulsions, microemulsions, solutions, suspensions, syrups, and elixirs. In addition to the active ingredient, the liquid dosage forms may contain inert diluents commonly used in the art, such as, for example (but not by way of limitation), water or other solvents, solubilizing agents, and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof. Besides inert diluents, the oral compositions can also include adjuvants such as (but not limited to) wetting agents, emulsifying and suspending agents, sweetening, flavoring, coloring, perfuming, and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

### EXAMPLES

Examples are provided hereinbelow. However, the present disclosure is to be understood as not limited in its application to the specific experimentation, results, and laboratory procedures disclosed below or elsewhere herein. Rather, each example is provided as one of various embodiments and are meant to be exemplary, not exhaustive. Tables 1-13 shown below provide a non-limiting list of D-amino acid peptides (D-peptides) which can be used in accordance with the methods and techniques of the present disclosure to inhibit the binding of GnRHR-AAbs to the GnRHR for the purposes and uses described elsewhere herein. In the tables, sequences which have at least one underscored amino acid symbol are to be read as having a deletion of that at least one amino acid. For example, the sequence “W W Q S F S C H T V C Q S F” shown in Table 1 is to be read as the sequence “Q S P S C H T V C Q S F” (SEQ ID NO:25) wherein the two (underscored) tryptophan residues have been deleted. Additionally, the present disclosure includes L-amino acid peptides (L-peptides) which comprise amino acid sequences which are the reverse of those shown in Tables 1-13 and comprise L-amino acids. For example, the reverse L-amino acid sequence of Q S F S C H T V C Q S F (SEQ ID NO:25) is F_{L} S_{L} Q_{L} C_{L} V_{L} T_{L} F_{L} C_{L} S_{L} F_{L} S_{L} Q_{L}, where the subscript _{L} represents an L-amino acid. The L-peptides of the present disclosure are particularly useful in assays for GnRHR-AAbs and diagnostic uses thereof where they are less susceptible to degradation by serum enzymes.

TABLE 1
D-Amino Acid Peptides of the Present Disclosure

| | | Comparison to SEQ ID NO: 1 |
|---|---|---|
| | | (underlined amino acids are |
| SEQ ID NO: | Amino Acid Sequence | deleted) |
| 2 | W W Q S F S C H T V C Q S F | W W Q S F S C H T V C Q S F |
| 3 | W W Q S F S C H T V C Q S | W W Q S F S C H T V C Q S F |
| 4 | W Q S F S C H T V C Q S F | W W Q S F S C H T V C Q S F |
| 5 | Q S F S C H T V C Q S | W W Q S F S C H T V C Q S F |
| 6 | W W Q S F S C H T V C Q F | W W Q S F S C H T V C Q S F |
| 7 | W Q S F S C H T V C Q F | W W Q S F S C H T V C Q S F |
| 8 | Q S F S C H T V C Q F | W W Q S F S C H T V C Q S F |
| 9 | W W Q S F S C H T V C S F | W W Q S F S C H T V C Q S F |
| 10 | W Q S F S C H T V C S F | W W Q S F S C H T V C Q S F |
| 11 | Q S F S C H T V C S F | W W Q S F S C H T V C Q S F |
| 12 | W W Q S F S C H T V C Q | W W Q S F S C H T V C Q S F |
| 13 | W Q S F S C H T V C Q | W W Q S F S C H T V C Q S F |
| 14 | Q S F S C H T V C Q | W W Q S F S C H T V C Q S F |
| 15 | W W Q S F S C H T V C S | W W Q S F S C H T V C Q S F |
| 16 | W Q S F S C H T V C S | W W Q S F S C H T V C Q S F |
| 17 | Q S F S C H T V C S | W W Q S F S C H T V C Q S F |
| 18 | W W Q S F S C H T V C F | W W Q S F S C H T V C Q S F |
| 19 | W Q S F S C H T V C F | W W Q S F S C H T V C Q S F |
| 20 | Q S F S C H T V C F | W W Q S F S C H T V C Q S F |
| 21 | W W Q S F S C H T V C | W W Q S F S C H T V C Q S F |
| 22 | W Q S F S C H T V C | W W Q S F S C H T V C Q S F |
| 23 | Q S F S C H T V C | W W Q S F S C H T V C Q S F |
| 24 | W Q S F S C H T V C Q S F | W W Q S F S C H T V C Q S F |
| 25 | Q S F S C H T V C Q S F | W W Q S F S C H T V C Q S F |

TABLE 2
D-Amino Acid Peptides of the Present Disclosure

| SEQ | | |
|---|---|---|
| ID | | Comparison to SEQ ID NO: 245 |
| NO: | Amino Acid Sequence | (underlined amino acids are deleted) |
| 26 | S F S C H T V C Q S F S F S C H T V C Q S | W W Q S F S C H T V C Q S F W W Q S F S C H |
| | | T V C Q S F |
| 27 | S F S C H T V C Q F S F S C H T V C S F | W W Q S F S C H T V C Q S F W W Q S F S C H |
| | | T V C Q S F |
| 28 | S F S C H T V C Q S F S C H T V C S | W W Q S F S C H T V C Q S F W W Q S F S C H |
| | | T V C Q S F |
| 29 | S F S C H T V C F S C H T V C Q S F | W W Q S F S C H T V C Q S F W W Q S F S C H |
| | | T V C Q S F |
| 30 | S C H T V C Q S F W F S C H T V C Q S F | W W Q S F S C H T V C Q S F W W Q S F S C H |
| | | T V C Q S F |
| 31 | W S C H T V C Q S F S S C H T V C Q S F | W W Q S F S C H T V C Q S F W W Q S F S C H |
| | | T V C Q S F |
| 32 | Q S C H T V C Q S F S C H T V C Q S F | W W Q S F S C H T V C Q S F W W Q S F S C H |
| | | T V C Q S F |
| 33 | Q F S C H T V C Q S F W W Q S F S H T V C Q S F | W W Q S F S C H T V C Q S F W W Q S F S C H |
| | | T V C Q S F |
| 34 | W W Q S F C H T V C Q S F W W Q S F H T V C Q S F | W W Q S F S C H T V C Q S F W W Q S F S C H |
| | | T V C Q S F |
| 35 | W Q S F S H T V C Q S F Q S F S H T V C Q S F | W W Q S F S C H T V C Q S F W W Q S F S C H |
| | | T V C Q S F |
| 36 | W Q S F C H T V C Q S F Q S F C H T V C Q S F | W W Q S F S C H T V C Q S F W W Q S F S C H |
| | | T V C Q S F |
| 37 | W Q S F H T V C Q S F Q S F H T V C Q S F | W W Q S F S C H T V C Q S F W W Q S F S C H |
| | | T V C Q S F |
| 38 | W W Q S F S T V C Q S F W W Q S F C T V C Q S F | W W Q S F S C H T V C Q S F W W Q S F S C H |
| | | T V C Q S F |

TABLE 3
D-Amino Acid Peptides of the Present Disclosure

| | | Comparison to SEQ ID NO: 1 |
|---|---|---|
| | | (underlined amino acids are |
| SEQ ID NO: | Amino Acid Sequence | deleted) |
| 39 | W W Q S F S H T V C Q S | W W Q S F S C H T V C Q S F |
| 40 | W W Q S F C H T V C Q S | W W Q S F S C H T V C Q S F |
| 41 | W W Q S F H T V C Q S | W W Q S F S C H T V C Q S F |
| 42 | W W Q S F S H T V C Q | W W Q S F S C H T V C Q S F |
| 43 | W W Q S F C H T V C Q | W W Q S F S C H T V C Q S F |
| 44 | W W Q S F H T V C Q | W W Q S F S C H T V C Q S F |
| 45 | W W Q S F S H T V C | W W Q S F S C H T V C Q S F |
| 46 | W W Q S F C H T V C | W W Q S F S C H T V C Q S F |
| 47 | W W Q S F H T V C | W W Q S F S C H T V C Q S F |
| 48 | W W Q S F S H T V C Q F | W W Q S F S C H T V C Q S F |
| 49 | W W Q S F C H T V C Q F | W W Q S F S C H T V C Q S F |
| 50 | W W Q S F H T V C Q F | W W Q S F S C H T V C Q S F |
| 51 | W W Q S F S H T V C S F | W W Q S F S C H T V C Q S F |
| 52 | W W Q S F C H T V C S F | W W Q S F S C H T V C Q S F |
| 53 | W W Q S F H T V C S F | W W Q S F S C H T V C Q S F |
| 54 | W W Q S F S H T V C F | W W Q S F S C H T V C Q S F |
| 55 | W W Q S F C H T V C F | W W Q S F S C H T V C Q S F |
| 56 | W W Q S F H T V C F | W W Q S F S C H T V C Q S F |
| 57 | W W Q S F S H T V C S | W W Q S F S C H T V C Q S F |
| 58 | W W Q S F C H T V C S | W W Q S F S C H T V C Q S F |
| 59 | W W Q S F H T V C S | W W Q S F S C H T V C Q S F |

TABLE 4
D-Amino Acid Peptides of the Present Disclosure

| | | Comparison to SEQ ID NO: 1 |
|---|---|---|
| | | (underlined amino acids are |
| SEQ ID NO: | Amino Acid Sequence | deleted) |
| 60 | W Q S F S H T V C Q S | W W Q S F S C H T V C Q S F |
| 61 | W Q S F C H T V C Q S | W W Q S F S C H T V C Q S F |
| 62 | W Q S F H T V C Q S | W W Q S F S C H T V C Q S F |
| 63 | W Q S F S H T V C Q | W W Q S F S C H T V C Q S F |
| 64 | W Q S F C H T V C Q | W W Q S F S C H T V C Q S F |
| 65 | W Q S F H T V C Q | W W Q S F S C H T V C Q S F |
| 66 | W Q S F S H T V C | W W Q S F S C H T V C Q S F |
| 67 | W Q S F C H T V C | W W Q S F S C H T V C Q S F |
| 68 | W Q S F H T V C | W W Q S F S C H T V C Q S F |
| 69 | W Q S F S H T V C Q F | W W Q S F S C H T V C Q S F |
| 70 | W Q S F C H T V C Q F | W W Q S F S C H T V C Q S F |
| 71 | W Q S F H T V C Q F | W W Q S F S C H T V C Q S F |
| 72 | W Q S F S H T V C S F | W W Q S F S C H T V C Q S F |
| 73 | W Q S F C H T V C S F | W W Q S F S C H T V C Q S F |
| 74 | W Q S F H T V C S F | W W Q S F S C H T V C Q S F |
| 75 | W Q S F S H T V C F | W W Q S F S C H T V C Q S F |
| 76 | W Q S F C H T V C F | W W Q S F S C H T V C Q S F |
| 77 | W Q S F H T V C F | W W Q S F S C H T V C Q S F |
| 78 | W Q S F S H T V C S | W W Q S F S C H T V C Q S F |
| 79 | W Q S F C H T V C S | W W Q S F S C H T V C Q S F |
| 80 | W Q S F H T V C S | W W Q S F S C H T V C Q S F |

TABLE 5
D-Amino Acid Peptides of the Present Disclosure

| | | Comparison to SEQ ID NO: 1 |
|---|---|---|
| | | (underlined amino acids are |
| SEQ ID NO: | Amino Acid Sequence | deleted) |
| 81 | Q S F S H T V C Q S | W W Q S F S C H T V C Q S F |
| 82 | Q S F C H T V C Q S | W W Q S F S C H T V C Q S F |
| 83 | Q S F H T V C Q S | W W Q S F S C H T V C Q S F |
| 84 | Q S F S H T V C Q | W W Q S F S C H T V C Q S F |
| 85 | W Q S F C H T V C Q | W W Q S F S C H T V C Q S F |
| 86 | Q S F H T V C Q | W W Q S F S C H T V C Q S F |
| 87 | Q S F S H T V C | W W Q S F S C H T V C Q S F |
| 88 | Q S F C H T V C | W W Q S F S C H T V C Q S F |
| 89 | Q S F H T V C | W W Q S F S C H T V C Q S F |
| 90 | Q S F S H T V C Q F | W W Q S F S C H T V C Q S F |
| 91 | Q S F C H T V C Q F | W W Q S F S C H T V C Q S F |
| 92 | Q S F H T V C Q F | W W Q S F S C H T V C Q S F |
| 93 | Q S F S H T V C S F | W W Q S F S C H T V C Q S F |
| 94 | Q S F C H T V C S F | W W Q S F S C H T V C Q S F |
| 95 | Q S F H T V C S F | W W Q S F S C H T V C Q S F |
| 96 | Q S F S H T V C F | W W Q S F S C H T V C Q S F |
| 97 | Q S F C H T V C F | W W Q S F S C H T V C Q S F |
| 98 | Q S F H T V C F | W W Q S F S C H T V C Q S F |
| 99 | Q S F S H T V C S | W W Q S F S C H T V C Q S F |
| 100 | Q S F C H T V C S | W W Q S F S C H T V C Q S F |
| 101 | Q S F H T V C S | W W Q S F S C H T V C Q S F |

TABLE 6
D-Amino Acid Peptides of the Present Disclosure

| | | Comparison to |
|---|---|---|
| | | SEQ ID NO: 1 (underlined amino |
| | | acids are deleted; a single W |
| | | or the W-W pair may be further |
| SEQ ID NO: | Amino Acid Sequence | deleted in alternate sequences) |
| 102 | W W Q S F S C H T V Q S F | W W Q S F S C H T V C Q S F |
| 103 | W W Q S F S C H T C Q S F | W W Q S F S C H T V C Q S F |
| 104 | W W Q S F S C H V C Q S F | W W Q S F S C H T V C Q S F |
| 105 | W W Q S F S C H T Q S F | W W Q S F S C H T V C Q S F |
| 106 | W W Q S F S C H V Q S F | W W Q S F S C H T V C Q S F |
| 107 | W W Q S F S C H C Q S F | W W Q S F S C H T V C Q S F |
| 108 | W W Q S F S C H Q S F | W W Q S F S C H T V C Q S F |
| 109 | W W Q S F S C T V C Q S F | W W Q S F S C H T V C Q S F |
| 110 | W W Q S F S C T V Q S F | W W Q S F S C H T V C Q S F |
| 111 | W W Q S F S C T C Q S F | W W Q S F S C H T V C Q S F |
| 112 | W W Q S F S C V C Q S F | W W Q S F S C H T V C Q S F |
| 113 | W W Q S F S C T Q S F | W W Q S F S C H T V C Q S F |
| 114 | W W Q S F S C V Q S F | W W Q S F S C H T V C Q S F |
| 115 | W W Q S F S C C Q S F | W W Q S F S C H T V C Q S F |
| 116 | W W Q S F S C Q S F | W W Q S F S C H T V C Q S F |

TABLE 7
D-Amino Acid Peptides of the Present Disclosure

| | | Comparison to SEQ ID NO: 1 |
|---|---|---|
| | | (underlined amino acids are |
| SEQ ID NO: | Amino Acid Sequence | deleted) |
| 117 | C Q S F | W W Q S F S C H T V C Q S F |
| 118 | V C Q S F | W W Q S F S C H T V C Q S F |
| 119 | T V C Q S F | W W Q S F S C H T V C Q S F |
| 120 | H T V C Q S F | W W Q S F S C H T V C Q S F |
| 121 | C H T V C Q S F | W W Q S F S C H T V C Q S F |
| 122 | S C H T V C Q S F | W W Q S F S C H T V C Q S F |
| 123 | F S C H T V C Q S F | W W Q S F S C H T V C Q S F |
| 124 | S F S C H T V C Q S F | W W Q S F S C H T V C Q S F |
| 125 | Q S F C | W W Q S F S C H T V C Q S F C |
| 126 | Q S F S C | W W Q S F S C H T V C Q S F |
| 127 | Q S F C | W W Q S F S C H T V C Q S F |
| 128 | W W Q S F S C H | W W Q S F S C H T V C Q S F |
| 129 | W Q S F S C H | W W Q S F S C H T V C Q S F |
| 130 | Q S F S C H | W W Q S F S C H T V C Q S F |
| 131 | W W Q S F C H | W W Q S F S C H T V C Q S F |
| 132 | W Q S F C H | W W Q S F S C H T V C Q S F |
| 133 | Q S F C H | W W Q S F S C H T V C Q S F |

TABLE 8
D-Amino Acid Peptides of the Present Disclosure

| | | Comparison to SEQ ID NO: 245 |
|---|---|---|
| | | (underlined amino acids are |
| SEQ | | deleted; a single W or the W-W |
| ID | | pair may be further deleted in |
| NO: | Amino Acid Sequence | alternate sequences) |
| 134 | W W Q S F S H T V Q S F W W Q S F C H T V Q S F | W W Q S F S C H T V C Q S F W W Q S F |
| | | S C H T V C Q S F |
| 135 | W W Q S F H T V Q S F W W Q S F S H T C Q S F | W W Q S F S C H T V C Q S F W W Q S F |
| | | S C H T V C Q S F |
| 136 | W W Q S F C H T C Q S F W W Q S F H T C Q S F | W W Q S F S C H T V C Q S F W W Q S F |
| | | S C H T V C Q S F |
| 137 | W W Q S F S H V C Q S F W W Q S F C H V C Q S F | W W Q S F S C H T V C Q S F W W Q S F |
| | | S C H T V C Q S F |
| 138 | W W Q S F H V C Q S F W W Q S F S T V C Q S F | W W Q S F S C H T V C Q S F W W Q S F |
| | | S C H T V C Q S F |
| 139 | W W Q S F C T V C Q S F W W Q S F T V C Q S F | W W Q S F S C H T V C Q S F W W Q S F |
| | | S C H T V C Q S F |
| 140 | W W Q S F S H T Q S F W W Q S F C H T Q S F | W W Q S F S C H T V C Q S F W W Q S F |
| | | S C H T V C Q S F |
| 141 | W W Q S F H T Q S F W W Q S F S H V C Q S F | W W Q S F S C H T V C Q S F W W Q S F |
| | | S C H T V C Q S F |
| 142 | W W Q S F C H V Q S F W W Q S F H V Q S F | W W Q S F S C H T V C Q S F W W Q S F |
| | | S C H T V C Q S F |
| 143 | W W Q S F S H C Q S F W W Q S F C H C Q S F | W W Q S F S C H T V C Q S F W W Q S F |
| | | S C H T V C Q S F |
| 144 | W W Q S F H C Q S F W W Q S F S H Q S F | W W Q S F S C H T V C Q S F W W Q S F |
| | | S C H T V C Q S F |
| 145 | W W Q S F C H Q S F W W Q S F H Q S F | W W Q S F S C H T V C Q S F W W Q S F |
| | | S C H T V C Q S F |
| 146 | W W Q S F S T V Q S F W W Q S F C T V Q S F | W W Q S F S C H T V C Q S F W W Q S F |
| | | S C H T V C Q S F |
| 147 | W W Q S F T V Q S F | W W Q S F S C H T V C Q S F |

TABLE 9
D-Amino Acid Peptides of the Present Disclosure

| | | Comparison to |
|---|---|---|
| | | SEQ ID NO: 1 (underlined amino |
| | | acids are deleted; a single W |
| | | or the W-W pair may be further |
| SEQ ID NO: | Amino Acid Sequence | deleted in alternate sequences) |
| 148 | W W Q S F S T C Q S F | W W Q S F S C H T V C Q S F |
| 149 | W W Q S F C T C Q S F | W W Q S F S C H T V C Q S F |
| 150 | W W Q S F T C Q S F | W W Q S F S C H T V C Q S F |
| 151 | W W Q S F S V C Q S F | W W Q S F S C H T V C Q S F |
| 152 | W W Q S F C V C Q S F | W W Q S F S C H T V C Q S F |
| 153 | W W Q S F V C Q S F | W W Q S F S C H T V C Q S F |
| 154 | W W Q S F S T Q S F | W W Q S F S C H T V C Q S F |
| 155 | W W Q S F C T Q S F | W W Q S F S C H T V C Q S F |
| 156 | W W Q S F T Q S F | W W Q S F S C H T V C Q S F |
| 157 | W W Q S F S V Q S F | W W Q S F S C H T V C Q S F |
| 158 | W W Q S F C V Q S F | W W Q S F S C H T V C Q S F |
| 159 | W W Q S F V Q S F | W W Q S F S C H T V C Q S F |
| 160 | W W Q S F S C Q S F | W W Q S F S C H T V C Q S F |
| 161 | W W Q S F C C Q S F | W W Q S F S C H T V C Q S F |
| 162 | W W Q S F C Q S F | W W Q S F S C H T V C Q S F |
| 163 | W W Q S F S Q S F | W W Q S F S C H T V C Q S F |
| 164 | W W Q S F C Q S F | W W Q S F S C H T V C Q S F |
| 165 | W W Q S F Q S F | W W Q S F S C H T V C Q S F |
| 166 | W W Q S F T V C Q S F | W W Q S F S C H T V C Q S F |

TABLE 10
D-Amino Acid Peptides of the Present Disclosure

| | | Comparison |
|---|---|---|
| | | to SEQ ID NO: 1 (underlined |
| | | amino acids are deleted; a |
| | | single W or the W-W pair, and/or |
| | | S₄, and/or F₅ may be further |
| SEQ ID NO: | Amino Acid Sequence | deleted in alternate sequences) |
| 167 | W W S F C H T V C Q S F | W W Q S F S C H T V C Q S F |
| 168 | W W S F S H T V C Q S F | W W Q S F S C H T V C Q S F |
| 169 | W W S F S C T V C Q S F | W W Q S F S C H T V C Q S F |
| 170 | W W S F S C H V C Q S F | W W Q S F S C H T V C Q S F |
| 171 | W W S F S C H T C Q S F | W W Q S F S C H T V C Q S F |
| 172 | W W S F S C H T V Q S F | W W Q S F S C H T V C Q S F |
| 173 | W W S F H T V C Q S F | W W Q S F S C H T V C Q S F |
| 174 | W W S F C T V C Q S F | W W Q S F S C H T V C Q S F |
| 175 | W W S F C H V C Q S F | W W Q S F S C H T V C Q S F |
| 176 | W W S F C H T C Q S F | W W Q S F S C H T V C Q S F |
| 177 | W W S F C H T V Q S F | W W Q S F S C H T V C Q S F |
| 178 | W W S F S T V C Q S F | W W Q S F S C H T V C Q S F |
| 179 | W W S F S H V C Q S F | W W Q S F S C H T V C Q S F |
| 180 | W W S F S H T C Q S F | W W Q S F S C H T V C Q S F |
| 181 | W W S F S H T V Q S F | W W Q S F S C H T V C Q S F |
| 182 | W W S F S C V C Q S F | W W Q S F S C H T V C Q S F |
| 183 | W W S F S C T C Q S F | W W Q S F S C H T V C Q S F |
| 184 | W W S F S C T V Q S F | W W Q S F S C H T V C Q S F |
| 185 | W W S F S C H C Q S F | W W Q S F S C H T V C Q S F |
| 186 | W W S F S C H V Q S F | W W Q S F S C H T V C Q S F |
| 187 | W W S F S C H T Q S F | W W Q S F S C H T V C Q S F |

TABLE 11
D-Amino Acid Peptides of the Present Disclosure

| | | Comparison to SEQ ID NO: 1 |
|---|---|---|
| | | (underlined amino acids are |
| | | deleted; a single W or the |
| | | W-W pair, and/or Q₃, and/or |
| | | F₅ may be further deleted |
| SEQ ID NO: | Amino Acid Sequence | in alternate sequences) |
| 188 | W W Q F C H T V C Q S F | W W Q S F S C H T V C Q S F |
| 189 | W W Q F S H T V C Q S F | W W Q S F S C H T V C Q S F |
| 190 | W W Q F S C T V C Q S F | W W Q S F S C H T V C Q S F |
| 191 | W W Q F S C H V C Q S F | W W Q S F S C H T V C Q S F |
| 192 | W W Q F S C H T C Q S F | W W Q S F S C H T V C Q S F |
| 193 | W W Q F S C H T V Q S F | W W Q S F S C H T V C Q S F |
| 194 | W W Q F H T V C Q S F | W W Q S F S C H T V C Q S F |
| 195 | W W Q F C T V C Q S F | W W Q S F S C H T V C Q S F |
| 196 | W W Q F C H V C Q S F | W W Q S F S C H T V C Q S F |
| 197 | W W Q F C H T C Q S F | W W Q S F S C H T V C Q S F |
| 198 | W W Q F C H T V Q S F | W W Q S F S C H T V C Q S F |
| 199 | W W Q F S T V C Q S F | W W Q S F S C H T V C Q S F |
| 200 | W W Q F S H V C Q S F | W W Q S F S C H T V C Q S F |
| 201 | W W Q F S H T C Q S F | W W Q S F S C H T V C Q S F |
| 202 | W W Q F S H T V Q S F | W W Q S F S C H T V C Q S F |
| 203 | W W Q F S C V C Q S F | W W Q S F S C H T V C Q S F |
| 204 | W W Q F S C T C Q S F | W W Q S F S C H T V C Q S F |
| 205 | W W Q F S C T V Q S F | W W Q S F S C H T V C Q S F |
| 206 | W W Q F S C H C Q S F | W W Q S F S C H T V C Q S F |
| 207 | W W Q F S C H V Q S F | W W Q S F S C H T V C Q S F |
| 208 | W W Q F S C H T Q S F | W W Q S F S C H T V C Q S F |

TABLE 12
D-Amino Acid Peptides of the Present Disclosure

| | | Comparison to SEQ ID NO: 1 |
|---|---|---|
| | | (underlined amino acids are |
| | | deleted; a single W or the |
| | | W-W pair, and one of Q₃ and |
| | | S4 may be further deleted |
| SEQ ID NO: | Amino Acid Sequence | in alternate sequences) |
| 209 | W W Q S C H T V C Q S F | W W Q S F S C H T V C Q S F |
| 210 | W W Q S S H T V C Q S F | W W Q S F S C H T V C Q S F |
| 211 | W W Q S S C T V C Q S F | W W Q S F S C H T V C Q S F |
| 212 | W W Q S S C H V C Q S F | W W Q S F S C H T V C Q S F |
| 213 | W W Q S S C H T C Q S F | W W Q S F S C H T V C Q S F |
| 214 | W W Q S S C H T V Q S F | W W Q S F S C H T V C Q S F |
| 215 | W W Q S H T V C Q S F | W W Q S F S C H T V C Q S F |
| 216 | W W Q S C T V C Q S F | W W Q S F S C H T V C Q S F |
| 217 | W W Q S C H V C Q S F | W W Q S F S C H T V C Q S F |
| 218 | W W Q S C H T C Q S F | W W Q S F S C H T V C Q S F |
| 219 | W W Q S C H T V Q S F | W W Q S F S C H T V C Q S F |
| 220 | W W Q S S T V C Q S F | W W Q S F S C H T V C Q S F |
| 221 | W W Q S S H V C Q S F | W W Q S F S C H T V C Q S F |
| 222 | W W Q S S H T C Q S F | W W Q S F S C H T V C Q S F |
| 223 | W W Q S S H T V Q S F | W W Q S F S C H T V C Q S F |
| 224 | W W Q S S C V C Q S F | W W Q S F S C H T V C Q S F |
| 225 | W W Q S S C T C Q S F | W W Q S F S C H T V C Q S F |
| 226 | W W Q S S C T V Q S F | W W Q S F S C H T V C Q S F |
| 227 | W W Q S S C H C Q S F | W W Q S F S C H T V C Q S F |
| 228 | W W Q S S C H V Q S F | W W Q S F S C H T V C Q S F |
| 229 | W W Q S S C H T Q S F | W W Q S F S C H T V C Q S F |

TABLE 13
D-Amino Acid Peptides of the Present Disclosure

| | | Comparison to SEQ ID NO: 245 |
|---|---|---|
| SEQ | | (underlined amino acids are deleted; a |
| ID | | single W, or the W-W pair, may be further |
| NO: | Amino Acid Sequence | deleted in alternate sequences) |
| 230 | W W C H T V C Q S F W W S H T V C Q S F | W W Q S F S C H T V C Q S F W W Q S F S C H T V |
| | | C Q S F |
| 231 | W W S C T V C Q S F W W S C H V C Q S F | W W Q S F S C H T V C Q S F. W W Q S F S C H T |
| | | V C Q S F |
| 232 | W W S C H T C Q S F W W S C H T V Q S F | W W Q S F S C H T V C Q S F W W Q S F S C H T V |
| | | C Q S F |
| 233 | W W H T V C Q S F W W C T V C Q S F | W W Q S F S C H T V C Q S F W W Q S F S C H T V |
| | | C Q S F |
| 234 | W W C H V C Q S F W W C H T C Q S F | W W Q S F S C H T V C Q S F W W Q S F S C H T V |
| | | C Q S F |
| 235 | W W C H T V Q S F W W S T V C Q S F | W W Q S F S C H T V C Q S F. W W Q S F S C H T |
| | | V C Q S F |
| 236 | W W S H V C Q S F W W S H T C Q S F | W W Q S F S C H T V C Q S F W W Q S F S C H T V |
| | | C Q S F |
| 237 | W W S H T V Q S F W W S C V C Q S F | W W Q S F S C H T V C Q S F W W Q S F S C H T V |
| | | C Q S F |
| 238 | W W S C T C Q S F W W S C T V Q S F | W W Q S F S C H T V C Q S F W W Q S F S C H T V |
| | | C Q S F |
| 239 | W W S C H C Q S F W W S C H V Q S F | W W Q S F S C H T V C Q S F W W Q S F S C H T V |
| | | C Q S F |
| 240 | W W S C H T Q S F W W S H T C Q S F | W W Q S F S C H T V C Q S F W W Q S F S C H T V |
| | | C Q S F |
| 241 | W W C H T C Q S F W W H T C Q S F | W W Q S F S C H T V C Q S F W W Q S F S C H T V |
| | | C Q S F |
| 242 | W W C H T V Q S F W W S H T V Q S F | W W Q S F S C H T V C Q S F W W Q S F S C H T V |
| | | C Q S F |
| 243 | W W H T V Q S F W W S H T Q S F | W W Q S F S C H T V C Q S F W W Q S F S C H T V |
| | | C Q S F |
| 244 | W W C H T Q S F W W H T Q S F | W W Q S F S C H T V C Q S F. W W Q S F S C H T |
| | | V C Q S F |

Further embodiments of the compositions and methods of the present disclosure are included in Appendix 1 of U.S. Provisional Application Ser. No. 62/969,880, filed Feb. 4, 2020, the entirety of which is incorporated by reference herein in its entirety.

Thus, in accordance with the present disclosure, there have been provided compositions, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A peptide comprising 4 to 45 amino acids, wherein the 4 to 45 amino acids comprise a D-amino acid sequence selected from at least one of SEQ ID NOS:1-245.

2. The peptide of claim 1, wherein the peptide binds to a gonadotropin releasing hormone receptor activating autoantibody (GnRHR-AAb) at an epitope which reduces binding of the GnRHR-AAb to an extracellular loop 2 (ECL2) of GnRHR.

3. A composition, comprising:
at least one peptide of claim 1 disposed in a pharmaceutically-acceptable carrier, vehicle, or diluent.

4. A composition, comprising:
at least one peptide of claim 1 conjugated to a labeling agent.

5. A drug delivery device or composition, comprising:
at least one peptide of claim 1; and
wherein the drug delivery device or composition substantially releases the one or more peptides over an extended period in a range of from about 6 hours to about 120 days.

6. A method of treating a disease or condition associated with gonadotropin releasing hormone receptor (GnRHR), the method comprising the step of:
administering to a subject in need of such therapy a D-amino acid peptide comprising 4 to 45 amino acids, wherein the 4 to 45 amino acids comprise a D-amino acid sequence selected from at least one of SEQ ID NOS:1-245.

7. The method of claim 6, wherein the D-amino acid peptide is administered in a composition comprising a pharmaceutically-acceptable carrier, vehicle, or diluent.

8. The method of claim 6, wherein the disease or condition is Polycystic Ovary Syndrome (PCOS).

9. The method of claim 6, wherein the disease is a condition related to GnRHR-AAb positive Metabolic Syndrome.

10. The method of claim 6, wherein the disease or condition is a refractory cancer selected from the group consisting of ovarian, breast, endometrial, uterine, prostate, and testicular cancer.

11. The method of claim 6, wherein the peptide is disposed in a drug delivery device or composition that substantially releases the peptide over an extended period in a range of from about 6 hours to about 120 days.

12. An assay for detecting a gonadotropin releasing hormone receptor activating autoantibody (GnRHR-AAb), comprising:
a test surface having a peptide bound thereto, the peptide comprising 4 to 45 amino acids, wherein the 4 to 45 amino acids comprise a D-amino acid sequence selected from at least one of SEQ ID NOS:1-245, or an L-amino acid reverse sequence thereof, or a portion of said D-amino acid sequence or L-amino acid sequence, which is able to bind with high affinity and specificity to the GnRHR-AAb.
